(19) United States
(12) Patent Application Publication
(10) Pub. No.: US 2019/0246919 A1
(43) Pub. Date: Aug. 15, 2019

(54) METHOD AND SYSTEM FOR CORRECTING PULSE TRANSIT TIME ASSOCIATED WITH ARTERIAL BLOOD PRESSURE OR BLOOD PRESSURE VALUE CALCULATED BY PULSE TRANSIT TIME

(71) Applicant: ZHEJIANG MAILIAN MEDICAL EQUIPMENT CO., LTD, Zhejiang (CN)

(72) Inventors: Yan CHEN, Chongqing (CN); Yu CHEN, Chongqing (CN)

(73) Assignee: ZHEJIANG MAILIAN MEDICAL EQUIPMENT CO., LTD, Zhejiang (CN)

(21) Appl. No.: 16/391,287

(22) Filed: Apr. 22, 2019

Related U.S. Application Data
(63) Continuation of application No. PCT/CN2017/111799, filed on Nov. 20, 2017.

(30) Foreign Application Priority Data
Nov. 22, 2016 (CN) 201611045054.5
Nov. 22, 2016 (CN) 201611046184.0

(51) Int. Cl. A61B 5/021; A61B 5/0285

(52) U.S. Cl. CPC A61B 5/02125; A61B 5/01; A61B 5/02116; A61B 5/0285

(57) ABSTRACT

The invention provides a method and a system for correcting pulse transit time (PTT) associated with arterial blood pressure or a blood pressure value (BP) calculated by the PTT, which are able to correct abnormal change of the PTT of a subject caused by cardiovascular diseases or various medical interventions or abnormal change of the BP calculated from the PTT. The invention real-timely detects pulse wave signals from the proximal end and distal end in each cardiac cycle, calculates the PTT and extracts one or more feature data and feature factors from the pulse wave signals; based on one or more feature factors, determines a cardiovascular state of the subject and a change in the state, and obtains one or more correction variables in each cardiac cycle; obtains a correction matrix based on the correction variable, and corrects the PTT associated with BP or the BP calculated by the PTT.

### CROSS-REFERENCE TO RELATED APPLICATION

This application is a continuation of international PCT application serial no. PCT/CN2017/111799, filed on Nov. 20, 2017, which claims the priority benefit of China application no. 201611045054.5, filed on Nov. 22, 2016 and China application serial no. 201611046184.0, filed on Nov. 22, 2016. The entirety of the above-mentioned patent application is hereby incorporated by reference herein and made a part of this specification.

### BACKGROUND OF THE INVENTION

### 1. Field of the Invention

The present invention relates to the technical field of arterial blood pressure measurement, and in particular to a method and a system for correcting pulse transit time (PTT) associated with arterial blood pressure or a blood pressure value calculated by PTT.

### 2. Description of Related Art

Existing methods and techniques for measuring blood pressure using pulse transit time or pulse wave velocity (PTT/PWV) require using conventional auscultation or oscillometric methods to measure one or more blood pressure values for initial calibration. The reason for calibration is that the relationship between PTT/PWV and blood pressure is object-dependent, that is, there is a definite relationship between PTT/PWV and blood pressure for each individual. The purpose of calibration is to determine such a functional relationship or to determine mathematical model parameters that are appropriate for the object.

However, the existing methods have certain limitations and can only be applied under conditions in which the human circulatory system does not have serious lesions and is not subject to outside intervention. Because only in the absence of lesions and intervention, the relationship between PTT/PWV and blood pressure is more regular for individuals, and may be described by a definite function or mathematical model. However, if the subject has cardiovascular disease, PTT/PWV will be affected by vascular disease, making the relationship between PTT/PWV and blood pressure seriously deviate from a conventional mathematical model, resulting in inaccurate blood pressure results as calculated. Besides, if the subject is being treated in a hospital (for example, undergoing surgery), the subject's circulatory system is affected by various medical interventions (such as transfusion, infusion, drug use, surgical operation, temperature change, etc.), PTT will undergo a series of abnormal changes. The calculation of blood pressure by using the abnormally changed PTT and an intrinsic mathematical model will produce a large error.

### SUMMARY OF THE INVENTION

In view of the deficiencies in the prior art, the present invention is directed to a method and a system for correcting the pulse transit time (PTT) associated with arterial blood pressure or a blood pressure value calculated by PTT, which are able to perform adaptive correction for the change of the PTT of a subject resulting from cardiovascular diseases or various medical interventions or the change of the blood pressure value calculated from the abnormally changed PTT.

The cardiovascular disease at least includes hypertension and arteriosclerosis.

The medical intervention at least includes the loss of blood and body fluid of the subject, the reduced blood volume of the subject resulting from food and liquid fasting, the increased blood volume of the subject resulting from transfusion and infusion, the use of antihypertensive drugs, antihypotensive drugs and positive inotropic drugs, surgical stimulation, endotracheal intubation, the increased body temperature of the subject, and the decreased body temperature of the subject.

In a first aspect, the present invention provides a method for correcting a PTT associated with arterial blood pressure or a blood pressure value calculated by PTT, including the following steps:

S1) selecting at least two body parts of a subject, and
acquiring the pulse wave signals simultaneously from the above body parts in each of a plurality of cardiac cycles;
S2) identifying feature data from each pulse wave signal in each cardiac cycle;
S3) extracting one or more pulse wave feature factors in each cardiac cycle according to the feature data, wherein the pulse wave feature factor is capable of indicating the cardiovascular state of the subject and a change in the cardiovascular state;
S4) determining the cardiovascular state of the subject and the change in the cardiovascular state based on one or more pulse wave feature factors, and then obtaining one or more correction variables in each cardiac cycle
S5) obtaining a correction matrix in each cardiac cycle based on one or more correction variables, and calculating an average correction matrix from a plurality of correction matrixes in a plurality of consecutive cardiac cycles; and
S6) correcting the abnormal change of a correction target by using the correction matrix or the average correction matrix; wherein the correction target is the PTT associated with arterial blood pressure or a blood pressure value calculated from the PTT.

In a second aspect, the present invention further provides a system for correcting a PTT associated with an arterial blood pressure, including:

a physiological signal acquisition unit, configured to real-timely acquire pulse wave signals from at least two body parts of a subject in each cardiac cycle, and other necessary signals for identifying the PTT;
a PTT identification unit, configured to calculate the PTT associated with the arterial blood pressure in each cardiac cycle;
a feature extraction unit, including:
a feature data identification module, configured to identify feature data of the pulse wave signal in each cardiac cycle; and
a pulse wave feature factor extraction module, configured to extract one or more pulse wave feature factors in each cardiac cycle;
and a correction unit, including:
a correction variable extraction module, configured to obtain one or more correction variables in each cardiac cycle according to the one or more pulse wave feature factors;
a correction matrix calculation module, configured to calculate a correction matrix in each cardiac cycle or an average correction matrix in a plurality of consecutive cardiac cycles according to the one or more correction variables obtained by the correction variable extraction module; and
a correction module, configured to correct the PTT by using the correction matrix.

Still further, the present invention provides a system for correcting a blood pressure value, where the blood pressure value is calculated by a PTT, and the system includes:

a physiological signal acquisition unit, configured to real-timely acquire pulse wave signals from at least two body parts of a subject in each cardiac cycle, and other necessary signals for identifying the PTT in real time;
a PTT identification unit, configured to calculate the PTT associated with an arterial blood pressure in each cardiac cycle;
a blood pressure calculation unit, configured to calculate a blood pressure value in each cardiac cycle according to the PTT obtained by the PTT identification unit;
a feature extraction unit, including:
a feature data identification module, configured to identify feature data of the pulse wave signal in each cardiac cycle; and
a pulse wave feature factor extraction module, configured to extract one or more pulse wave feature factors in each cardiac cycle; and
a correction unit, including:
a correction variable extraction module, configured to obtain one or more correction variables in each cardiac cycle according to the one or more pulse wave feature factors;
a correction matrix calculation module, configured to calculate a correction matrix in each cardiac cycle or an average correction matrix in a plurality of consecutive cardiac cycles according to the one or more correction variables obtained by the correction variable extraction module; and
a correction module, configured to correct the blood pressure value calculated from the PTT by using the correction matrix.

The system is a computer program product, the computer program product includes a computer readable code, and when executed by a suitable computer or processor, the computer readable code is configured to instruct a computer or processor to execute the method described above.

The present invention has the following beneficial effects:

The present invention provides the method and the system for correcting the PTT associated with arterial blood pressure or the blood pressure calculated by PTT, which are able to correct abnormal change of the PTT of the subject caused by cardiovascular diseases or various medical interventions, or t the blood pressure calculated by the abnormally changed PTT. The correction method and system according to the present invention combining with the existing mathematical model may continuously measure the arterial blood pressure in each cardiac cycle under clinical conditions with high accuracy.

### BRIEF DESCRIPTION OF THE DRAWINGS

FIG. 1 is a flow chart of a method provided by the present invention for correcting pulse transit time (PTT) associated with arterial blood pressure or a blood pressure value calculated by PTT.

FIG. 2 is a schematic diagram of a first arterial blood pressure measurement system based on PTT in an embodiment of the present invention.

FIG. 3 is a schematic diagram of a second arterial blood pressure measurement system based on PTT in an embodiment of the present invention.

### DESCRIPTION OF THE EMBODIMENTS

In order to more clearly understand the above objects, features and advantages of the present invention, the present invention will be further described in detail below with reference to the drawings and embodiments.

In the description of the present invention, the terms “the first”, “the second” and the like are used for the purpose of description only and are not to be construed as indicating or implying relative importance.

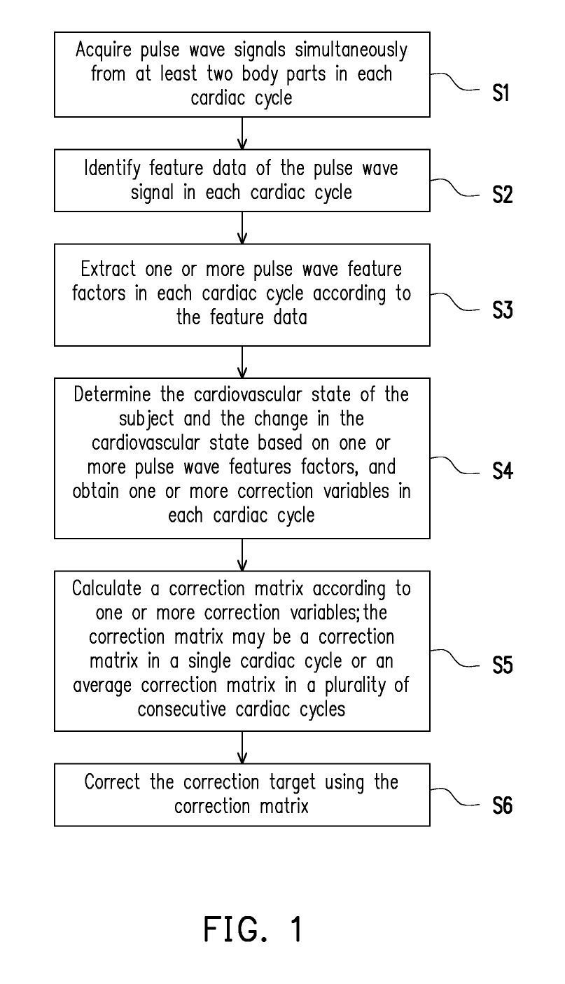

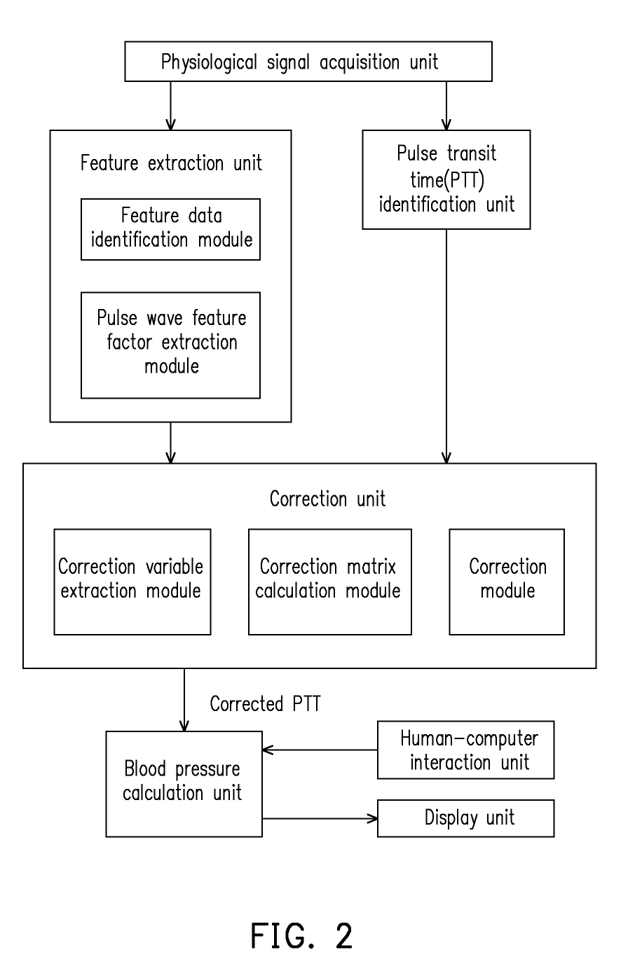

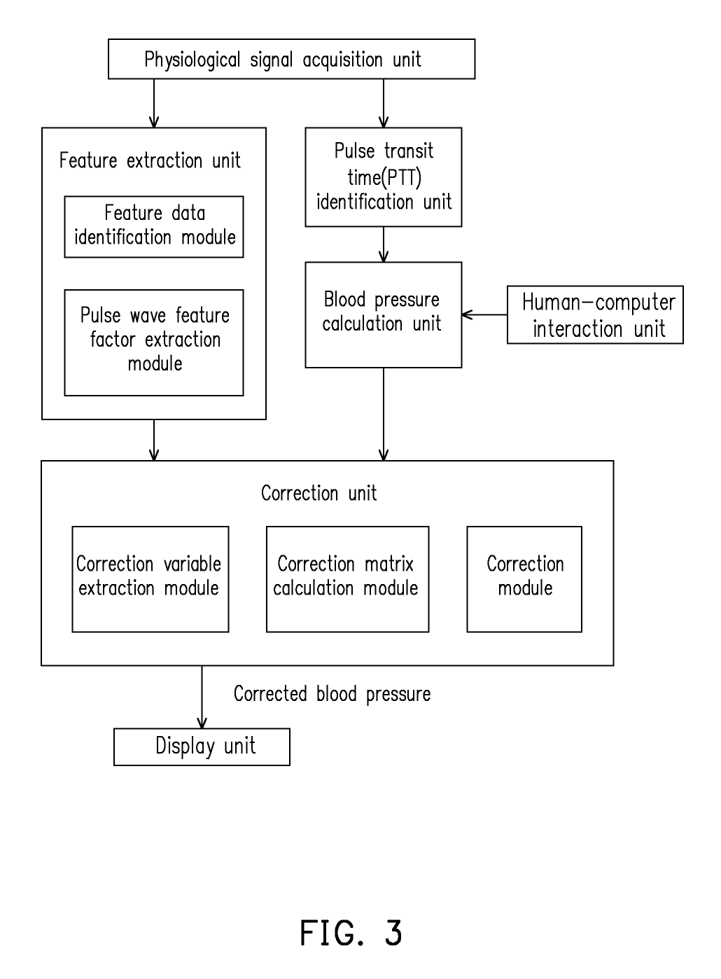

FIG. 2 and FIG. 3 show two arterial blood pressure measurement systems based on pulse transit time (PTT), respectively, both of which function based on the correction method provided in FIG. 1.

Among them, a physiological signal acquisition unit in each of the measurement systems shown in FIG. 2 and FIG. 3 is used in step S1 of FIG. 1, and is used to acquire pulse wave signals from at least two body parts of a subject, and other signals required to identify PTT (for example, signals of electrocardiogram (ECG) and impedance cardiogram (ICG)) in real time. The human body part at which the pulse wave of a proximal end is acquired is preferably the ear, and the human body part at which the pulse wave of a distal end is acquired is preferably the toe. The sensor for detecting the pulse wave signal is preferably an infrared photoplethysmograph (PPG), and the sensor for detecting the ECG may be a plurality of skin electrodes.

A PTT identification unit is used to calculate the PTT associated with arterial blood pressure.

On the one hand, PTT may be obtained by synchronously measuring ECG, PPG and ICG. At first, pulse arrival time (PAT) is obtained from ECG and PPG, in which the PAT is defined as a time interval between an R-wave peak of the ECG and the starting point of a pulse wave signal detected by PPG. Pre-ejection period (PEP) is then determined by ECG and ICG, and PTT is equal to PAT minus PEP.

On the other hand, preferably, the PTT may also be obtained by synchronously detecting one PPG signal from proximal end and the other from distal end and calculating a time difference between the two pulse wave signals. A time difference between starting points of the two pulse wave signals may be regarded as the PTT associated with diastolic blood pressure (DBP) denoted as T_{d}, and a time difference between the wave peaks of the two pulse wave signals may be regarded as the PTT associated with systolic blood pressure (SBP) denoted as Tₛ. The specific identification methods for T_{d} and Tₛ may be understood by reference to the literature Yan C H E N, Changyun W E N, Guocai T A O, and Min B I, Continuous and Noninvasive Measurement of Systolic and Diastolic Blood Pressure by One Mathematical Model with the Same Model Parameters and Two Separate Pulse Wave Velocities.

A feature extraction unit includes a feature data identification module and a pulse wave feature factor extraction module. The feature data identification module is used in step S2 of FIG. 1, which is configured to identify feature data of the pulse wave signal in each cardiac cycle.

The feature data in step S2 includes a height of the aortic valve closing point on the pulse wave of the proximal artery, that is, a height at a junction of the systolic phase and the diastolic phase (indicated by a symbol h_{sd}); the systolic time of a pulse wave of the proximal artery (indicated by a symbol tₛ); the diastolic time of the pulse wave of the proximal artery (indicated by a symbol t_{d}); the maximum height of the pulse wave of the proximal artery (indicated by a symbol hₘₐₓ); the systolic time of the pulse wave of the distal artery (indicated by a symbol tₛ₋ₜₒₑ); the diastolic time of the pulse wave of the distal artery (indicated by a symbol t_{d-toe}); the maximum height of the pulse wave of the distal artery (indicated by the symbol hₘₐₓ₋ₜₒₑ); the time interval between the starting point of the pulse wave of the distal artery and the midpoint of the wave peak (indicated by a symbol t_{ch-toe}), in which the midpoint of the wave peak refers to a midpoint of the raising edge turning point and the falling edge turning point on the pulse wave, and the definition may be understood by reference to the literature Yan C H E N, Changyun W E N, Guocai T A O, and Min B I, Continuous and Noninvasive Measurement of Systolic and Diastolic Blood Pressure by One Mathematical Model with the Same Model Parameters and Two Separate Pulse Wave Velocities; the time interval between the starting point of the pulse wave of the distal artery and the highest point of the wave peak (indicated by a symbol tₘₐₓ₋ₜₒₑ); and the amplitude of the pulse wave of the proximal and distal arteries in a longitudinal-axis direction (indicated by a symbol h).

The pulse wave feature factor extraction module is used in step S3 of FIG. 1, which is configured to extract one or more pulse wave feature factors in each cardiac cycle, which includes: a first factor indicating that the subject is in hypotensive state or his/her blood pressure is decreasing; a second factor and a third factor indicating that the subject is in hypertension state or his/her blood pressure is increasing; a fourth factor and a fifth factor indicating the blood volume state and the temperature state of the subject; and a sixth factor, a seventh factor, an eighth factor, and a ninth factor indicating the peripheral vasodilating state of the subject.

A correction unit includes a correction variable extraction module, a correction matrix calculation module and a correction module. The correction variable extraction module is used in step S4 of FIG. 1, which determines the cardiovascular state of the subject and the change in the cardiovascular state based on one or more pulse wave features factors, and obtains one or more correction variables in each cardiac cycle.

When the subject exhibits the state of hypotension or reduced blood pressure due to loss of blood and body fluids, food and liquid fasting, use of anesthetic drugs, vasodilator drugs, diuretic-antihypertensive drugs, sympathetic inhibitors, renin-angiotensin inhibitors, calcium antagonists or other reasons, the above state can be determined by the first pulse wave feature factor, and a first variable a₁ is obtained; when the subject exhibits the state of hypertension or changing from normotension to hypertension due to hypertensive disease, arteriosclerosis, use of vasopressor, vasoconstrictor or positive inotropic drugs, surgical stimulation, endotracheal intubation or other reasons, the above state can be determined by the first, second and third pulse wave feature factors, and a second variable a₂ is obtained; when the pulse waveform of the proximal or distal artery is varied due to surgical operation interference or other reasons, or the subject exhibits a change in blood volume due to loss of blood and body fluid, food and liquid fasting, transfusion, infusion or other reasons, or a change in skin temperature of the body part at where the pulse wave signals are obtained due to increased body temperature, decreased body temperature or other reasons, the above changes of state can be determined by the first, second, third, fourth, and fifth pulse wave feature factors, and a third variable a₃ is obtained; and when the subject exhibits a state of dilated distal artery due to the use of anesthetic drugs, vasodilator drugs, an increase in body temperature or other reasons, the above state can be determined by the sixth and seventh pulse wave feature factors, and a fourth variable a₄ and a fifth variable a₅ are obtained; when the subject exhibits a state that the distal artery dilates more than the proximal artery due to the use of anesthetic drugs, vasodilators, an increase in body temperature or other reasons, the above state can be determined by the first, eighth and ninth pulse wave feature factors, and a sixth variable a₆ and a seventh variable a₇ are obtained.

In addition, in the mathematical formula of each variable, d₁, d₁₋₂, d₂, d₃, d₃₋₂, c₄, d₄, c₅, d₅, d₆, d₇, d₅, and d₉ are all preset thresholds for determining the states, and the specific value of the thresholds are determined according to the specific mode and human body location at which the pulse wave signal is obtained, and the correction target.

In a preferred embodiment, for the pulse wave signals obtained by PPG from the posterior auricular artery and the toe artery, the preset thresholds are:

when the correction target is the PTT associated with SBP denoted as Tₛ or the SBP calculated from Tₛ: d₁=0.76 to 0.84, d₁₋₂=1.04 to 1.12, and d₂=1.17 to 1.27;
when the correction target is the PTT associated with DBP denoted as T or the DBP calculated from T_{d}: d₁=0.74 to 0.82, d₁₋₂=0.98 to 1.06, and d₂=1.33 to 1.43.

When the correction target is PTT (including Tₛ and T_{d}) or blood pressure value calculated from the PTT (including SBP and DBP): c₄=(d₄+(age—14)/8)/100, d₄=23 to 35, c₅=(d₅+(age—14)/8)/100, d₅=27 to 39, d₆=0.97 to 1.03, d₇=1.52 to 1.58, d₈=1.42 to 1.48, d₃₋₂=1.21 to 1.31, d₃=0.02 to 0.14, and d₉=0.67 to 0.73.

The correction matrix calculation module is used in step S5 of the correction method of FIG. 1, which is configured to calculate a correction matrix according to one or more correction variables obtained by the correction variable extraction module; the correction matrix may be a correction matrix in a single cardiac cycle or an average correction matrix in a plurality of consecutive cardiac cycles.

The correction module is used in step S6 of the correction method of FIG. 1, which is configured to correct the correction target using the correction matrix. As shown in FIG. 2, the correction target may be PTT, including Tₛ and T_{d}. And as shown in FIG. 3, the correction target may also be the blood pressure value calculated from PTT, including SBP and DBP.

The blood pressure calculation unit shown in FIG. 2 and FIG. 3 is configured to calculate the continuous beat-to-beat blood pressure value according to a mathematical model between the PTT or the pulse wave velocity (PWV) and the blood pressure. Many academic papers report the mathematical model which continuously measures beat-to-beat blood pressure using PTT/PWV, and a specific method for constructing the model may be understood by reference to the following literatures: Yan C H E N, Changyun W E N, Guocai T A O, Min B I, and Guoqi L I, A Novel Modeling Methodology of the Relationship Between Blood Pressure and Pulse Wave Velocity; Yan C H E N, Changyun W E N, Guocai T A O, and Min B I, Continuous and Noninvasive Measurement of Systolic and Diastolic Blood Pressure by One Mathematical Model with the Same Model Parameters and Two Separate Pulse Wave Velocities; Younhee C H O I, Qiao Z H A N G, Seokbum K O, Noninvasive cuffless blood pressure estimation using pulse transit time and Hilbert-Huang transfonnrm; Zheng Y, Poon C C, Yan B P, Lau J Y, Pulse Arrival Time Based Cuff-Less and 24-H Wearable Blood Pressure Monitoring and its Diagnostic Value in Hypertension; Mukkamala R, Hahn J O, Inan O T, Mestha L K, Kim C S, Töreyin H, Kyal S, Toward Ubiquitous Blood Pressure Monitoring via Pulse Transit Time: Theory and Practice.

The blood pressure calculation unit may calculate the blood pressure value using the PTT according to the disclosed mathematical model. In an exemplary embodiment, the blood pressure calculation unit involves the following calculation steps.

Step 1: calculate PWV associated with SBP denoted as Vₛ and PWV associated with DBP denoted as V_{d} by the following method:

$${V}_{s} = \frac{L}{{T}_{sma}} and {V}_{d} = \frac{L}{{T}_{dma}} ;$$

where, L is a distance that the pulse wave travels through, that is, a distance between two pulse wave detection points, which may be obtained by measurement. Tₛₘₐ is the corrected Tₛ in a plurality of cardiac cycles, and T_{dma} is the corrected T_{d} in a plurality of cardiac cycles;
Step 2: calculate the SBP and the DBP by the following methods:

$$SBP = {b}_{ij} {e}^{- \frac{{k}_{ij}}{{V}_{s}}} and DBP = {b}_{ij} {e}^{- \frac{{k}_{ij}}{{V}_{d}}} ;$$

where, kᵢⱼ and bᵢⱼ are model parameters for populations at different ages and genders, i represents age and i=1, 2, . . . , n, n≤100; j represents gender and j=M/F, M represents model parameters of male, and F represents model parameters of female. kᵢⱼ and bᵢⱼ may be determined by statistical data of large sample populations, and kᵢⱼ and bᵢⱼ may also be determined based on reference measurement results (or calibration values) of SBP and DBP of the measured subject.

A human-computer interaction unit shown in FIG. 2 and FIG. 3 provides a human-machine interface for a user to input basic physiological parameters (such as age, gender, height, weight, etc.) and a reference measurement result (or calibration value) of blood pressure for determining a model parameter of the mathematical model in the blood pressure calculation unit.

The display unit shown in FIG. 2 and FIG. 3 is configured to display the calculated blood pressure value in real time and provide an alarm when blood pressure crosses the line.

Next, an example will be given to illustrate how the correction method and the arterial blood pressure measurement system of the present invention are applied clinically: a non-invasive continuous beat-to-beat blood pressure measurement was performed on one patient during an anesthesia process, and then changes in PTT were adaptively corrected.

The patient was male, with the age of 40 years old, the height of 170 cm and the weight of 60 kg.

The patient was first measured using the arterial blood pressure measurement system of FIG. 2, namely, PPG signals of posterior auricular artery and toe artery were obtained by two photoelectric sensors, PTT was obtained using the PTT identification unit, and a blood pressure value in each cardiac cycle was calculated using the blood pressure calculation unit according to a preferred mathematical model combined with the calculation method.

When the patient receives anesthesia induction, the patient's peripheral arterial blood vessels rapidly expanded under the action of anesthetic drugs, resulting in a decrease in systemic blood pressure and an increase in PTT. However, the effect of anesthetic drugs on PTT was higher than that on blood pressure for this patient. The degree of PTT increase was much greater than the degree of blood pressure reduction. The blood pressure value calculated using excessively increased PTT and the preferred mathematical model was lower than the blood pressure value obtained using a standard method (such as auscultation). At this time, it is necessary to correct the excessive increase in PTT by using the first variable a₁, the fourth variable a₄, and the fifth variable a₅.

In a certain cardiac cycle, the first pulse wave feature factor k_{sd-m-0}=0.9 was obtained; for the Ts, the preset thresholds d₁ was 0.8 and d₁₋₂ was 1.08; for T_{d}, d₁ was 0.78, and d₁₋₂ was 1.02; at this time, d₁≤k_{sd-m-0}≤d₁₋₂, indicating the insufficient power of the pulse wave propagation, and the first variable a₁=(d₁₋₂−k_{sd-m-0})×0.50=(1.08−0.9)×0.50=0.09 for Ts; a₁=(d₁₋₂−k_{sd-m-0})×0.4=(1.02−0.9)×0.4=0.048 for T_{d}. In the cardiac cycle, the obtained sixth pulse wave feature factor kₛ₋ₜ₋ₜₒₑ=0.82>0.8, indicating the dilation of the distal artery, and the fourth variable a₄=kₛ₋ₜ₋ₜₒₑ−0.8=0.82−0.8=0.02; the seventh pulse wave feature factor kₛ₋ₘ₋ₜₒₑ=0.74, the preset threshold d₉ was 0.7, and the fifth variable a₅=kₛ₋ₘ₋ₜₒₑ−d₉=0.74−0.7=0.04.

Then, the correction matrix in the cardiac cycle was calculated:

For Tₛ, A=Σᵢ₌₁³aᵢ=a₁+a₄+a₅=0.09+0.02+0.04=0.15; the correction matrixes in eight cardiac cycles were obtained by the same method: A₁=0.15, A₂=0.17, A₃=0.18, A₃=0.16, A₅=0.14, A₆=0.12, A₇=0.16, and A₈=0.14; the average correction matrix in 8 consecutive cardiac cycles was calculated: Aₘ=⅛Σⱼ₌₁⁸Aⱼ=⅛(A₁+A₂+A₃+A₄+A₅+A₆+A₇+A₈)=⅛(0.15+0.17+0.18+0.16+0.14+0.12+0.16+0.14)=0.153.

For T_{d}, A=Σᵢ₌₁³aᵢ=a₁+a₄+a₅=0.048+0.02+0.04=0.108; the correction matrixes in eight cardiac cycles were obtained by the same method: A₁=0.108, A₂=0.112, A₃=0.114, A₃=0.111, A₅=0.107, A₆=0.104, A₇=0.106, and A₈=0.108; the average correction matrix in 8 consecutive cardiac cycles was calculated: Aₘ=⅛Σⱼ₌₁⁸Aⱼ=⅛(A₁+A₂+A₃+A₄+A₅+A₆+A₇+A₈)=⅛(0.108+0.112+0.114+0.111+0.107+0.104+0.106+0.108)=0.108.

Tₛ (milliseconds) in eight cardiac cycles was obtained using PTT identification unit according to the preferred calculation method of PTT mentioned above: Tₛ₁=151, Tₛ₂=153, Tₛ₃=154, Tₛ₄=158, Tₛ₅=152, Tₛ₆=148, Tₛ₇=146, Tₛ₈=144, Tₛₘ=⅛Σⱼ₌₁⁸Tₛⱼ=⅛(Tₛ₁+Tₛ₂+Tₛ₃+Tₛ₄+Tₛ₅+Tₛ₆+Tₛ₇+Tₛ₈)=⅛(151+153+154+158+152+148+146+144)=151; and the Tₛₘ after correction was Tₛₘₐ=Tₛₘ(1−Aₘ)=151×(1−0.153)=128.

T_{d} (milliseconds) in eight cardiac cycles was obtained: T_{d1}=226, T_{d2}=228, T_{d3}=231, T_{d4}=227, T_{d5}=225, T_{d6}=222, T_{d7}=218, T_{d8}=221, T_{dm}=⅛Σⱼ₌₁⁸T_{dj}=⅛(T_{d1}+T_{d2}+T_{d3}+T_{d4}+T_{d5}+T_{d6}+T_{d7}+T_{d8})=⅛ (226+228+231+227+225+222+218+221)=225; the T_{dm} after correction was T_{dma}=T_{dm} (1−Aₘ)=225×(1−0.108)=201.

The Vₛ (m/s) and V_{d} (m/s) were calculated according to the preferred method in the blood pressure calculation unit. The patient was 1.7 meters tall and L was a distance from an ear detection point to a toe detection point, which was 1.4 meters.

The PWV that was not corrected by the method of the present invention was:

$${V}_{s} = \frac{L}{{T}_{sm}} = \frac{1.4}{151} \times 1000 = 9.3 ; {V}_{d} = \frac{L}{{T}_{d m}} = \frac{1.4}{225} \times 1000 = 6.2 ;$$

but the PWV that was corrected by the method of the present invention was:

$${V}_{s} = \frac{L}{{T}_{sma}} = \frac{1.4}{128} \times 1000 = 10.9 ; {V}_{d} = \frac{L}{{T}_{dma}} = \frac{1.4}{201} \times 1000 = 7.0 .$$

The SBP and DBP were calculated according to the preferred mathematical model mentioned above. For men around 40 years old, the statistical values of model parameters kᵢⱼ and bᵢⱼ (i=40,j=M) from large sample populations were 11.2 and 255, respectively.

The SBP and DBP that were not corrected by the method of the present invention were calculated as:

$$SBP = 255 \times {e}^{- \frac{11.2}{9.3}} = 76 and DBP = 255 \times {e}^{- \frac{11.2}{6.2}} = 42 ;$$

The SBP and DBP that were corrected by the method of the present invention were calculated as:

$$SBP = 255 \times {e}^{- \frac{11.2}{10.9 }} = 91 and DBP = 255 \times {e}^{- \frac{11.2}{7.0}} = 51 ;$$

At the same time, the SBP obtained by auscultation (standard method) was 90, and the DBP obtained by auscultation (standard method) was 55. Obviously, in the presence of an anesthetic drug, the blood pressure value calculated from the uncorrected PTT was lower than the blood pressure value obtained by the standard method, but the blood pressure value calculated from the PTT corrected using the method of the present invention was much closer to the blood pressure value obtained by the standard method. It is shown that the correction method of the present invention combined with the existing mathematical model may continuously measure the arterial blood pressure in each cardiac cycle in the case of medical intervention with high accuracy.

What is claimed is:

1. A method for correcting a pulse transit time associated with arterial blood pressure or a blood pressure value calculated from the pulse transit time, wherein the method comprises the following steps:
S1) selecting at least two body parts of a subject, and acquiring the pulse wave signals simultaneously from the above body parts in each of a plurality of cardiac cycles;
S2) identifying feature data from each pulse wave signal in each cardiac cycle;
S3) extracting one or more pulse wave feature factors in each cardiac cycle based on the feature data, wherein the pulse wave feature factor is capable of indicating the cardiovascular state of the subject and a change in the cardiovascular state;
S4) determining the cardiovascular state of the subject and the change in the cardiovascular state based on one or more pulse wave feature factors, and then obtaining one or more correction variables in each cardiac cycle;
S5) obtaining a correction matrix in each cardiac cycle based on one or more correction variables, and calculating an average correction matrix from a plurality of correction matrixes in a plurality of consecutive cardiac cycles; and
S6) correcting the change of a correction target by using the correction matrix or the average correction matrix; wherein the correction target is the pulse transit time associated with the arterial blood pressure or a blood pressure value calculated from the.

2. The method according to claim 1, wherein the pulse wave signal at least comprises one pulse wave signal of a proximal artery and one pulse wave signal of a distal artery, wherein the proximal artery may be a carotid artery, a thoracic aorta, a brachial artery, a superficial temporal artery or a posterior auricular artery, and the distal artery may be a radial artery, a finger artery, an arteria dorsalis pedis or a toe artery.

3. The method according to claim 2, wherein the feature data in the step S2 comprises at least one of the followings: a height of an aortic valve closing point on a pulse wave of the proximal artery, that is, a height at a junction of a systolic phase and a diastolic phase denoted as h_{s,d}; a systolic time of a pulse wave of the proximal artery denoted as tₛ; a diastolic time of the pulse wave of the proximal artery denoted as t_{d}; the maximum height of the pulse wave of the proximal artery denoted as hₘₐₓ; the systolic time of the pulse wave of the distal artery denoted as tₛ₋ₜₒₑ; the diastolic time of the pulse wave of the distal artery denoted as t_{d-toe}; the maximum height of the pulse wave of the distal artery denoted as hₘₐₓ₋ₜₒₑ; the time interval between the starting point of the pulse wave of the distal artery and the midpoint of the wave peak denoted as t_{ch-toe}, in which the midpoint of the wave peak refers to a midpoint of the raising edge turning point and the falling edge turning point on the wave peak; the time interval between the starting point of the pulse wave of the distal artery and the highest point of the wave peak denoted as tₘₐₓ₋ₜₒₑ; and the amplitude of the pulse wave of the proximal and distal arteries in a longitudinal-axis direction denoted as h.

4. The method according to claim 3, wherein the pulse wave feature factor in the step S3 comprises at least one of the followings:
a first factor k_{sd-m-0} indicating that the subject is in a hypotensive state or blood pressure is decreasing, which is obtained by the following method: obtaining a ratio of h_{sd} to an average height in the systolic phase on the pulse wave of the proximal artery
$${k}_{sd \text{-} m \text{-} 0} = \frac{{t}_{s} {h}_{sd}}{{\int }_{0}^{{t}_{s}} hdt} ,$$
wherein the systolic phase refers to a pulse wave segment before the aortic valve closing point in a direction of a time axis;
a second factor and a third factor indicating that the subject is in a hypertension state or blood pressure is increasing, which are obtained by the following methods:
the second factor k_{sd-m-ts}: obtaining a ratio of h_{sd} to an average height in a partial segment of the diastolic phase on the pulse wave of the proximal artery
$${k}_{sd \text{-} m \text{-} ts} = \frac{{t}_{s} {h}_{sd}}{{\int }_{{t}_{s}}^{2 {t}_{s}} hdt} ,$$
wherein the partial segment of the diastolic phase refers to a pulse wave segment between the points at tₛ and 2 times of tₛ in the direction of the time axis;
the third factor k_{sd-m-2}: obtaining a ratio of h_{sd} to the average height of the partial segment of the entire pulse wave of the proximal artery
$${k}_{sd \text{-} m \text{-} 2} = \frac{2 {t}_{s} {h}_{sd}}{{\int }_{0}^{2 {t}_{s}} hdt} ,$$
wherein the partial segment of the entire pulse wave refers to a pulse wave segment between the starting point and the point at 2 times of tₛ in the direction of the time axis;
a fourth factor and a fifth factor indicating the blood volume state and the temperature state of a subject, which are obtained by the following methods:
the fourth factor k_{d-m-t}_{_{d}}: obtaining a ratio of the average height of the diastolic phase to hₘₐₓ on the pulse wave of the proximal artery
$${k}_{d \text{-} m \text{-} {t}_{d}} = \frac{{\int }_{{t}_{s}}^{{t}_{s} + {t}_{d}} htd}{{t}_{d} {h}_{m ax}} ,$$
wherein the diastolic phase refers to a pulse wave segment after the aortic valve closing point in the direction of the time axis;
the fifth factor k_{d-m-t}_{_{d-toe}}: obtaining a ratio of the average height in the diastolic phase to the maximum height hₘₐₓ₋ₜₒₑ of the pulse wave on the pulse wave of the distal artery
$${k}_{d \text{-} m \text{-} {t}_{d \text{-} toe}} = \frac{{\int }_{{t}_{s \text{-} toe}}^{{t}_{s \text{-} toe} + {t}_{d \text{-} toe}} hdt}{{t}_{d \text{-} toe} {h}_{ma x \text{-} toe}} ,$$
wherein the diastolic phase refers to a pulse wave segment after the aortic valve closing point in the direction of the time axis;
a sixth factor, a seventh factor, an eighth factor, and a ninth factor indicating a peripheral vasodilating state of the subject, which are obtained by the following methods:
the sixth factor kₛ₋ₜ₋ₜₒₑ: obtaining a ratio of the time interval between the starting point and the wave peak to the systolic phase of the pulse wave of the distal artery
$${k}_{s \text{-} t \text{-} toe} = \frac{{t}_{m ax \text{-} toe} + {t}_{ch \text{-} toe}}{2 {t}_{s \text{-} toe}} or {k}_{s \text{-} t \text{-} toe} = \frac{{t}_{ma x \text{-} toe}}{{t}_{s \text{-} toe}} ,$$
wherein the peak may be the highest point of the wave peak or the average of the highest point and the midpoint of the wave peak;
the seventh factor kₛ₋ₘ₋ₜₒₑ: obtaining a ratio of the average height of the systolic phase of the pulse wave of the distal artery to hₘₐₓ₋ₜₒₑ
$${k}_{s \text{-} m \text{-} toe} = \frac{{\int }_{0}^{{t}_{s \text{-} toe}} hdt}{{t}_{s \text{-} toe} {h}_{ma x \text{-} toe}} ;$$
the eighth factor kₛ₋ₘ₋ₜₒₑ₋ₑₐᵣ: obtaining a ratio of a pulse wave systolic phrase area of the distal artery to a pulse wave systolic phrase area of the proximal artery
$${k}_{s \text{-} m \text{-} toe \text{-} ear} = \frac{{\int }_{0}^{{t}_{s \text{-} toe}} hdt}{{\int }_{0}^{{t}_{s}} hdt} ;$$
and
the ninth factor kₜₛ₋ₜₒₑ₋ₑₐᵣ: obtaining a ratio of pulse wave systolic time of the distal artery to a pulse wave systolic time of the proximal artery
$${k}_{ts \text{-} toe \text{-} ear} = \frac{{t}_{s \text{-} toe}}{{t}_{s}} .$$

5. The method according to claim 4, wherein the correction variable in the step S4 comprises a first variable a₁, and an applicable condition of the first variable a₁ is: hypotension or blood pressure reduction due to various reasons;
the first variable a₁ is obtained by the following method:
determining whether the subject is in a hypotension state or a state in which blood pressure is decreasing according to the first pulse wave feature factor k_{sd-m-0};
if d₁≤k_{sd-m-0}≤d₁₋₂, indicating that the blood pressure is significantly reduced and the power of pulse wave propagation is insufficient, wherein in the case that the correction target is the pulse transit time associated with systolic blood pressure, a₁=(d₁₋₂−k_{sd-m-0})×0.50, in the case that the correction target is the pulse transit time associated with diastolic blood pressure, a₁=(d₁₋₂−k_{sd-m-0})×0.4;
if k_{sd-m-0}<d₁, indicating that the blood pressure drops to a very low level and the power of pulse wave propagation is seriously insufficient, wherein in the case that the correction target is the pulse transit time associated with systolic blood pressure, a₁=28×0.50, in the case that the correction target is the pulse transit time associated with diastolic blood pressure, a₁=0.24×0.5;
if k_{sd-m-0}>d₁₋₂, indicating that the blood pressure is not significantly reduced, and the power of pulse wave propagation is sufficient, then a₁=0;
wherein, d₁ and d₁₋₂ are preset thresholds for determining the state.

6. The method according to claim 4, wherein the correction variable in step S4 comprises a second variable a₂, and an applicable condition of the second variable a₂ is: hypertension and a change from normotention to hypertension due to various reasons;
the second variable a₂ is obtained by the following method:
determining whether the subject is in a state of hypertension or a state of blood pressure increasing from normotention to hypertension according to the first pulse wave feature factor k_{sd-m-0}, the second factor k_{sd-m-ts}, and the third factor k_{sd-m-2};
if |k_{sd-m-0}−k_{sd-m-ts}|≥40 and (k_{sd-m-0}+k_{sd-m-ts})/2≥k_{sd-m-2}, indicating that the feature of the diastolic phase of a proximal arterial pulse wave is abnormally changed, then k_{sd-m}=2×k_{sd-m-2}−(k_{sd-m-0}+k_{sd-m-ts})/2;
otherwise k_{sd-m}=k_{sd-m-2};
if k_{sd-m}>(d₂+(age—14)/15/100), indicating that the blood pressure is already very high or is rising rapidly, and in this state, the power corresponding to the highest blood pressure is insufficient, wherein in the case that the correction target is the pulse transit time associated with systolic blood pressure, a₂=k_{sd-m}−(d₂+(age—14)/15/100), in the case that the correction target is the pulse transit time associated with diastolic blood pressure, a₂=(k_{sd-m}−(d₂+(age—14)/15/100))×0.5;
if k_{sd-m}≤(d₂+(age—14)/15/100), indicating that blood pressure is not high or has an unobvious increasing trend, and in this state, the power corresponding to the highest blood pressure is sufficient, then a₂=0;
wherein, k_{sd-m}, is an intermediate variable, age is age and d₂ is a preset threshold for determining the state.

7. The method according to claim 4, wherein the correction variable in the step S4 comprises a third variable a₃, and an applicable condition of the third variable a₃ is: a pulse waveform variation of the proximal or distal artery occurs, and a change in blood volume or a change in skin temperature of the body part at where the pulse wave signal is detected;
the third variable a₃ is obtained by the following method:
determining whether the pulse wave of the proximal or distal artery undergoes waveform variation and whether the blood volume of the subject or the skin temperature at the source of the pulse wave signal is changed according to the first factor k_{sd-m-0}, the second factor k_{sd-m-ts}, the third factor k_{sd-m-2}, the fourth factor k_{d-m-t}_{_{d}}, and the fifth factor k_{d-m-t}_{_{d-toe}};
if k_{sd-m-ts}≤d₃₋₂, indicating that the early diastolic phase of the pulse waveform of the proximal artery is abnormally increased, in which case a supplementary correction requires to be performed for k_{d-m-t}_{_{d}}, and a correction result is recorded as k_{d-m-t}_{_{d}}₋₁, then k_{d-m-t}_{_{d}}₋1=k_{d-m-t}_{_{d}}−(d₃₋₂−k_{sd-m-ts})×75/100;
if k_{d-m-t}_{_{d}}≤d₃, indicating that the pulse waveform of the proximal artery is abnormally changed, in which case k_{d-m-t}_{_{d}} requires to be corrected and a correction result is recorded as k_{d-m-t}_{_{d}}₋₁, then k_{d-m-t}_{_{d}}₋₁=d₃;
if k_{d-m-t}_{_{d-toe}}≤d₃, indicating that the pulse waveform of the distal artery is abnormally changed, in which case k_{d-m-t}_{_{d-toe}} is required to be corrected, then k_{d-m-t}_{_{d-toe}}=d₃;
then obtaining the average values of the two similar factors on the proximal arterial and the distal arterial pulse waves, denoted as k_{d-m-a}, which is obtained by the following methods:
k_{d-m-a}=(k_{d-m-t}_{_{d}}₋₁+k_{d-m-t}_{_{d-toe}})/2;
if |k_{sd-m-0}−k_{sd-m-ts}|≥40 and (k_{sd-m-0}+k_{sd-m-ts})/2≥k_{sd-m-2} and k_{sd-m-ts}≥d₃₋₂, indicating that the diastolic phase of the proximal and distal arterial pulse waveforms are abnormally changed, in which case k_{d-m-a} requires to be corrected, then k_{d-m-a}=(k_{d-m-t}_{_{d}}₋₁+k_{d-m-t}_{_{d-toe}}+(k_{sd-m-0}+k_{sd-m-ts})/2−k_{sd-m-2})/2,
otherwise k_{d-m-a}=(k_{d-m-t}_{_{d}}₋₁+k_{d-m-t}_{_{d-toe}})/2;
if c₄<k_{d-m-a}<c₅, indicating that the blood volume of the subject is normal, and the skin temperature of body part at where the pulse wave signal is detected is also normal, then a₃=0;
if k_{sd-m-0}<d₆ or k_{sd-m-2}>d₇, indicating that the blood volume of the subject is very low or the blood pressure is extremely high, and information about the diastolic phase is unstable at this time, then a₃=0;
if k_{sd-m-0}≥d₆+0.10 and k_{sd-m-2}≤d₈ and k_{d-m-a}≤c₄, indicating that in a normal blood pressure state, the blood volume of the subject decreases or the skin temperature of body part at where the pulse wave signal is detected decreases, then a₃=(c₄−k_{d-m-a})×67/100;
if
$$\{ \begin{matrix}{d}_{6} \leq {k}_{sd \text{-} m \text{-} 0} < {d}_{6} + 0.10 \\ {k}_{d \text{-} m \text{-} a} \leq {c}_{4}\end{matrix} or \{ \begin{matrix}{d}_{8} < {k}_{sd \text{-} m \text{-} 2} < {d}_{7} \\ {k}_{d \text{-} m \text{-} a} \leq {c}_{4}\end{matrix} ,$$
indicating that in a low or high blood pressure state, the blood volume of the subject decreases or the skin temperature of body part at where the pulse wave signal is detected decreases, then a₃=(c₄−k_{d-m-a})×50/100;
if k_{sd-m-0}≥d₆+0.10 and k_{sd-m-2}≤d₈ and k_{d-m-a}≥c₅, indicating that in a normal blood pressure state, the blood volume of the subject increases or the skin temperature of body part at where the pulse wave signal is detected increases, then a₃=(c₅−k_{d-m-a})×62/100; and
if
$$\{ \begin{matrix}{d}_{6} \leq {k}_{sd \text{-} m \text{-} 0} < {d}_{6} + 0.10 \\ {k}_{d \text{-} m \text{-} a} \leq {c}_{5}\end{matrix} or \{ \begin{matrix}{d}_{8} < {k}_{sd \text{-} m \text{-} 2} < {d}_{7} \\ {k}_{d \text{-} m \text{-} a} \leq {c}_{5}\end{matrix} ,$$
indicating that in a low or high blood pressure state, the blood volume of the subject increases or the skin temperature of body part at where the pulse wave signal is detected increases, then a₃=(c₅−k_{d-m-a})×45/100;
wherein c₄, d₄, c₅, d₅, d₆, d₇, d₈, d₃₋₂ and d₃ are preset thresholds for determining the state.

8. The method according to claim 4, wherein the correction variable in the step S4 comprises a fourth variable a₄, and an applicable condition of the fourth variable a₄ is: distal arteriectasis due to various reasons;
the fourth variable a₄ is obtained by the following method:
determining whether the distal artery is dilated according to the sixth factor kₛ₋ₜ₋ₜₒₑ;
if tₘₐₓ₋ₜₒₑ≥t_{ch-toe}, indicating that the highest point of the pulse wave peak of the distal artery is behind the midpoint of the pulse wave peak, in which case kₛ₋ₜ₋ₜₒₑ requires to be corrected, then
$${k}_{s \text{-} t \text{-} toe} = \frac{{t}_{ma x \text{-} toe} + {t}_{ch \text{-} toe}}{{t}_{s \text{-} toe}} ,$$
otherwise
$${k}_{s \text{-} t \text{-} toe} = \frac{{t}_{ma x \text{-} toe}}{{t}_{s \text{-} toe}} ,$$
if kₛ₋ₜ₋ₜₒₑ>0.8, indicating that the distal artery is dilated, then a₄=kₛ₋ₜ₋ₜₒₑ−0.8; and
if kₛ₋ₜ₋ₜₒₑ≤0.8, indicating that the distal artery is not obviously dilated, then a₄=0.

9. The method according to claim 4, wherein the correction variable in the step S4 comprises a fifth variable a₅, and an applicable condition of the fifth variable a₅ is a distal arteriectasia due to various reasons; and
the fifth variable a₅ is obtained by the following method:
determining whether the distal artery is dilated according to the sixth factor kₛ₋ₜ₋ₜₒₑ and the seventh factor kₛ₋ₘ₋ₜₒₑ;
if kₛ₋ₘ₋ₜₒₑ<d₉, indicating that the distal artery is not obviously dilated, then a₅=0;
if kₛ₋ₘ₋ₜₒₑ≥d₉ and kₛ₋ₜ₋ₜₒₑ≥0.8, indicating that the distal artery dilatation is very obvious, then a₅=kₛ₋ₘ₋ₜₒₑ−d₉; and
if kₛ₋ₘ₋ₜₒₑ≥d₉ and kₛ₋ₜ₋ₜₒₑ<0.8, indicating that the distal artery is in a certain degree of dilatation, then a₅=(kₛ₋ₘ₋ₜₒₑ−d₉)/2;
wherein d₉ is a preset threshold for determining the state.

10. The method according to claim 4, wherein the correction variable in the step S4 comprises a sixth variable a₆, and an applicable condition of the sixth variable a₆ is: a dilatation degree of the distal artery exceeds that of the proximal artery due to various reasons;
the sixth variable a₆ is obtained by the following method:
determining whether the dilatation degree of the distal artery exceeds that of the proximal artery according to the first factor k_{sd-m-0} and the eighth factor kₛ₋ₘ₋ₜₒₑ₋ₑₐᵣ;
if kₛ₋ₘ₋ₜₒₑ₋ₑₐᵣ<1.0, indicating that the systolic phrase area of the distal arterial pulse wave is smaller than that of the proximal arterial pulse wave, and the distal end artery has no obvious dilatation comparing to the proximal artery, then a₆=0;
when kₛ₋ₘ₋ₜₒₑ₋ₑₐᵣ>1.08, indicating that the systolic phrase area of the distal arterial pulse wave is much greater than that of the proximal arterial pulse wave, then c₆=1.08,
at this time, if tₛ>220 and k_{sd-m-0}>0.88, indicating that the feature of the proximal arterial pulse wave is normal, then a₆=c₆−1.0;
if tₛ<160 or k_{sd-m-0}<0.80, indicating that the feature of the proximal arterial pulse wave is seriously abnormally changed, then a₆=(c₆−1.0)×0.34; and
if 160<tₛ≤220 or 0.80<k_{sd-m-0}≤0.88, indicating that the feature of the proximal arterial pulse wave is abnormally changed but not very serious, then a₆=(c₆−1.0)×0.67;
when 1.0≤kₛ₋ₘ₋ₜₒₑ₋ₑₐᵣ≤1.08, indicating that the systolic phrase area of the distal arterial pulse wave is slightly greater than that of the proximal arterial pulse wave, then c₆=kₛ₋ₘ₋ₜₒₑ₋ₑₐᵣ−1.0;
at this time, if tₛ>220 and k_{sd-m-0}>0.88, indicating that the feature of the proximal arterial pulse wave is normal, then a₆=c₆,
if t≤160 or k_{sd-m-0}≤0.80, indicating that the feature of the proximal arterial pulse wave is seriously abnormally changed, then a₆=c₆×0.34,
if 160<tₛ≤220 or 0.80<k_{sd-m-0}≤0.88, indicating that the feature of the proximal arterial pulse wave is abnormally changed but not very serious, then a₆=c₆×0.67;
wherein c₆ is an intermediate variable.

11. The method according to claim 4, wherein the correction variable in the step S4 comprises a seventh variable a₇, and an applicable condition of the seventh variable a₇ is a dilatation degree of the distal artery exceeds that of the proximal artery due to various reasons; and
the seventh variable a₇ is obtained by the following method:
determining whether the distal artery is dilated according to the first factor k_{sd-m-0} and the ninth factor kₜₛ₋ₜₒₑ₋ₑₐᵣ;
if kₜₛ₋ₜₒₑ₋ₑₐᵣ<1.0, indicating that the systolic time of the distal arterial pulse wave is shorter than that of the proximal arterial pulse wave, indicating that the distal artery has no obvious dilatation comparing to the proximal artery, then a₇=0;
when kₜₛ₋ₜₒₑ₋ₑₐᵣ>1.08, indicating that the systolic time of the distal arterial pulse wave is much longer than that of the proximal arterial pulse wave, then c₇=1.08,
at this time, if tₛ>220 and k_{sd-m-0}>0.88, indicating that the feature of the proximal arterial pulse wave is normal, then a₇=c₇−1.0;
if tₛ<160 or k_{sd-m-0}<0.80, indicating that the feature of the proximal arterial pulse wave is seriously abnormally changed, then a₇=(c₇−1.0)×0.34;
if 160<tₛ≤220 or 0.80<k_{sd-m-0}≤0.88, indicating that the feature of the proximal arterial pulse wave is abnormally changed but not very serious, then a₇=(c₇−1.0)×0.67;
when 1.0≤kₜₛ₋ₜₒₑ₋ₑₐᵣ≤1.08, indicating that the systolic time of the distal arterial pulse wave is slightly longer than that of the proximal arterial pulse wave, then c₇=kₜₛ₋ₜₒₑ₋ₑₐᵣ−1.0;
at this time, if tₛ>220 and k_{sd-m-0}>0.88, indicating that the feature of the proximal arterial pulse wave is normal, then a₇=c₇,
if tₛ≤160 or k_{sd-m-0}≤0.80, indicating that the feature of the proximal arterial pulse wave is seriously abnormally changed, then a₇=c₇×0.34;
if 160<tₛ≤220 or 0.80<k_{sd-m-0}≤0.88, indicating that the feature of the proximal arterial pulse wave is abnormally changed but not very serious, then a₇=c₇×0.67; and
wherein, c₇ is an intermediate variable.

12. The method according to claim 1, wherein the correction matrix of one cardiac cycle in the step S5 is obtained by the following method:
A=Σᵢ₌₁^{N}aᵢ;
wherein aᵢ is the ith correction variable;
the average correction matrix of a plurality of consecutive cardiac cycles in step S5 is obtained by the following method:
$${A}_{m} = \frac{1}{N} {\sum }_{j = 1}^{N} {A}_{j}$$
wherein Aⱼ is the correction matrix of the jth cardiac cycle.

13. The method according to claim 12, wherein the pulse transit time associated with the arterial blood pressure in the step S6 comprises a pulse transit time associated with a diastolic blood pressure T_{d} and a pulse transit time associated with a systolic blood pressure Tₛ;
the pulse transit time associated with the systolic blood pressure Tₛ is corrected by the following method:
$${T}_{sa} = {T}_{s} ( 1 - A ) ;$$
$$or$$
$${T}_{sm a} = {T}_{sm} ( 1 - {A}_{m} ) and {T}_{sm} = \frac{1}{N} {\sum }_{j = 1}^{N} {T}_{sj} ,$$
wherein, Tₛₐ is the corrected Tₛ in a single cardiac cycle, and Tₛₘₐ is the corrected Tₛ in a plurality of cardiac cycles; Tₛₘ is the averaged Tₛ in N cardiac cycles; and Tₛⱼ is the Tₛ in the jth cardiac cycle;
the pulse transit time associated with the diastolic blood pressure is corrected by the following method:
$${T}_{da} = {T}_{d} ( 1 - A ) ;$$
$$or$$
$${T}_{d ma} = {T}_{d m} ( 1 - {A}_{m} ) and {T}_{d m} = \frac{1}{N} {\sum }_{j = 1}^{N} {T}_{dj} ,$$
wherein, T_{da} is the corrected T_{d} in a single cardiac cycle, and Tama is the corrected T_{d} in a plurality of cardiac cycles; T_{dm} is averaged T_{d} in N cardiac cycles; and T_{dj} is the T_{d} in the jth cardiac cycle.

14. The method according to claim 12, wherein the blood pressure value calculated from the pulse transit time in the step S6 refers to a blood pressure value calculated by using the pulse transit time or a pulse wave velocity through various mathematical models or function relationships, which comprises a systolic blood pressure and a diastolic blood pressure;
the systolic blood pressure is corrected by the following method:
$${SBP}_{a} = SBP ( 1 - A ) ;$$
$$or$$
$${SBP}_{ma} = {SBP}_{m} ( 1 - {A}_{m} ) and {SBP}_{m} = \frac{1}{N} {\sum }_{j = 1}^{N} {SBP}_{j} ,$$
wherein SBP is the systolic blood pressure before being corrected, SBPₐ is the corrected systolic blood pressure in a single cardiac cycle, and SBPₘₐ is the corrected systolic blood pressure in a plurality of cardiac cycles; SBPₘ is averaged systolic blood pressure in N cardiac cycles before being corrected; and SBPⱼ is the systolic blood pressure in the jth cardiac cycle before being corrected;
the diastolic blood pressure is corrected by the following method:
$${DBP}_{a} = DBP ( 1 - A ) ;$$
$$or$$
$${DBP}_{ma} = {DBP}_{m} ( 1 - {A}_{m} ) and {DBP}_{m} = \frac{1}{N} {\sum }_{j = 1}^{N} {DBP}_{j} ,$$
wherein DBP is the diastolic blood pressure before being corrected, DBPₐ is the corrected diastolic blood pressure in a single cardiac cycle, and DBPₘₐ is the corrected diastolic blood pressure in a plurality of cardiac cycles; DBPₘ is averaged diastolic blood pressure in N cardiac cycles before being corrected; and DBPⱼ is diastolic blood pressure in the jth cardiac cycle before being corrected.

15. The method according to claim 1, wherein a manner of obtaining the pulse wave signal in the step S1 comprises any one or more of the followings: a pressure sensor, a photoplethysmograph or an impedance plethysmography.

16. A system for correcting a pulse transit time associated with an arterial blood pressure, wherein the system comprises:
a physiological signal acquisition unit, configured to real-timely acquire pulse wave signals from at least two body parts of a subject in each cardiac cycle, and other necessary signals for identifying the pulse transit time;
a pulse transit time identification unit, configured to calculate the pulse transit time associated with the arterial blood pressure in each cardiac cycle;
a feature extraction unit, comprising:
a feature data identification module, configured to identify feature data of the pulse wave signal in each cardiac cycle; and
a pulse wave feature factor extraction module, configured to extract one or more pulse wave feature factors in each cardiac cycle; and
a correction unit, comprising:
a correction variable extraction module, configured to obtain one or more correction variables in each cardiac cycle according to the one or more pulse wave feature factors;
a correction matrix calculation module, configured to calculate a correction matrix in each cardiac cycle or an average correction matrix in a plurality of consecutive cardiac cycles according to the one or more correction variables obtained by the correction variable extraction module; and
a correction module, configured to correct the pulse transit time by using the correction matrix.

17. A system for correcting a blood pressure value, wherein the blood pressure value is calculated by a pulse transit time, wherein the system comprises:
a physiological signal acquisition unit, configured to real-timely acquire pulse wave signals from at least two body parts of a subject in each cardiac cycle, and other necessary signals for identifying the pulse transit time;
a pulse transit time identification unit, configured to calculate the pulse transit time associated with an arterial blood pressure in each cardiac cycle;
a blood pressure calculation unit, configured to calculate the blood pressure value in each cardiac cycle according to the pulse transit time obtained by the pulse transit time identification unit;
a feature extraction unit, comprising:
a feature data identification module, configured to identify feature data of the pulse wave signal in each cardiac cycle; and
a pulse wave feature factor extraction module, configured to extract one or more pulse wave feature factors in each cardiac cycle; and
a correction unit, comprising:
a correction variable extraction module, configured to obtain one or more correction variables in each cardiac cycle according to the one or more pulse wave feature factors;
a correction matrix calculation module, configured to calculate a correction matrix in each cardiac cycle or an average correction matrix in a plurality of consecutive cardiac cycles according to the one or more correction variables obtained by the correction variable extraction module; and
a correction module, configured to correct the blood pressure value calculated from the pulse transit time by using the correction matrix.
